(19) United States
(12) Patent Application Publication
(10) Pub. No.: US 2012/0142870 A1
(43) Pub. Date: Jun. 7, 2012

(54) Isocyanate- and Aldimine Group- Carrying Compounds with a Low Isocyanate Monomer Content

(75) Inventor: Urs BURCKHARDT, Zurich (CH)

(73) Assignee: SIKA TECHNOLOGY AG, Zurich (CH)

(21) Appl. No.: 13/370,926

(22) Filed: Feb. 10, 2012

Related U.S. Application Data
(62) Division of application No. 12/450,181, filed on Sep. 15, 2009.

(30) Foreign Application Priority Data
Mar. 27, 2007 (EP) 07105003.3

(51) Int. Cl. C08G 18/83; C07C 263/16

(52) U.S. Cl. 525/454; 560/336; 525/453

(57) ABSTRACT

Isocyanate- and aldimine group-carrying compounds VB of formula (I), their compositions and I uses thereof. The moisture cross-linking polyurethane compositions are characterized by a surprisingly low content in monomeric isocyanates and are therefore especially suitable as hot-melt adhesives.

### RELATED APPLICATIONS

This application is a divisional of U.S. patent application Ser. No. 12/450,181, filed Sep. 15, 2009, which is a National Phase application of PCT Application Number PCT/EP2008/053633, filed Mar. 27, 2008, which claims priority from EP Patent Application Number 07105003.3, filed Mar. 27, 2007.

### TECHNICAL FIELD

The invention relates to the field of aldimines and of the moisture-crosslinking polyurethanes, and to the use thereof, especially as reactive hotmelt adhesives with a low content of monomeric diisocyanates.

### STATE OF THE ART

Aldimines are condensation products of amines and aldehydes, and constitute a substance class which has been known for some time. On contact with water, aldimines can be hydrolyzed to the corresponding amines and aldehydes, whereas they are stable in the absence of water. Owing to this property, they can be used as a bound or protected form of amines. For example, aldimines are used in polyurethane chemistry, where they serve as moisture-activable crosslinkers, so-called “latent amines” or “latent hardeners”, for isocyanate-containing polyurethane polymers. The use of aldimines as latent hardeners in moisture-curing isocyanate-containing systems has two advantages: first, the formation of undesired gas bubbles in the cured polymer can be avoided, since the curing proceeds via the latent amine—in contrast to the direct reaction of the isocyanate with moisture—without release of carbon dioxide (CO₂); secondly, higher curing rates can be achieved. However, their use also harbors difficulties. For example, the storage stability of certain aldimines together with isocyanates can be inadequate. Moreover, the systems require a relatively large amount of water for curing, and the aldehyde released in the course of curing can cause an undesired odor.

U.S. Pat. No. 4,469,831, U.S. Pat. No. 4,853,454, U.S. Pat. No. 5,087,661 and WO 2004/13200 disclose moisture-curing polyurethane compositions comprising polyaldimines, which have a good storage stability, and the systems from WO 2004/13200 additionally cure without odor.

In addition, there are attempts to use, instead of polyaldimines and polyisocyanates, compounds which have both isocyanate groups and aldimino groups. Such compounds are self-curing on contact with moisture, significantly less water being required for curing than in the case of curing of polyisocyanates with polyaldimines. For example, U.S. Pat. No. 6,136,942 describes polyurethane polymers which have both isocyanate groups and aldimino groups and which are obtainable from the reaction of polymers having isocyanate groups with monoaldimines which additionally possess an active hydrogen in the form of a secondary amino group. The monoaldimines usable for this purpose are, however, difficult to obtain, which greatly limits formulation latitude. Moreover, these compositions have problems with lightfastness in cured form.

A further difficulty in the case of use of polyurethane polymers having isocyanate groups is the problem of the monomeric diisocyanates. In the reaction of polyols with diisocyanates to give, or in the preparation of, oligomeric polyisocyanates, owing to the random distribution of the possible reaction products, a residual content of unconverted monomeric diisocyanates remains in the polymer formed. These monomeric diisocyanates, also referred to as “isocyanate monomers” for short, are volatile compounds and can be harmful owing to their irritant, allergenic and/or toxic action. They are therefore undesirable in many fields of use. This is especially true of spray applications and of compositions to be processed while hot, for example hotmelt adhesives.

Various ways of lowering the proportion of monomeric diisocyanates in polyurethane polymers having isocyanate groups have been described. For example, the monomeric diisocyanates can be partially or completely removed subsequently, for example by extraction or distillation, from the polyurethane polymer having isocyanate groups, which, however, is inconvenient and therefore costly. A low NCO/OH ratio in the preparation of polyurethane polymers having isocyanate groups leads directly to a low isocyanate monomer content; however, polymers prepared in this way have, owing to oligomerization reactions (“chain extension”), an increased viscosity and are therefore generally more difficult to process and less storage-stable. The use of an asymmetric diisocyanate with two isocyanate groups of different reactivity, as described, for example, in WO 03/006521, likewise leads directly to a low content of monomeric diisocyanates. However, the polyurethane polymers having isocyanate groups obtainable in this way generally have slow crosslinking, since principally only the less reactive of the two isocyanate groups is available for the crosslinking reaction.

### SUMMARY OF THE INVENTION

It is therefore an object of the present invention to provide isocyanate systems which overcome the disadvantages of the prior art.

This object can surprisingly be achieved by a compound having isocyanate and aldimino groups as claimed in claim 1.

This compound, which relates to a first aspect of the invention, can be prepared easily and is storage-stable in the absence of water. Selected embodiments (u≧v in formula (I)) are self-curing under the influence of moisture.

Further aspects of the present invention relate to a process as claimed in claim 11 for preparing said compound and to compositions as claimed in claim 14 which, as well as said compound, comprise at least one polymer P.

Further aspects of the present invention relate to a cured composition as claimed in claim 20, to the use of the composition as an adhesive as claimed in claim 21, and to a process for adhesive bonding as claimed in claim 22 and to the articles formed therefrom as claimed in claim 25.

These compositions are storable, contain a low content of monomeric diisocyanates and crosslink rapidly with moisture and without bubble formation. The compound having aldimino groups is obtainable from the partial reaction of polyisocyanates with asymmetric dialdimines and water. In a preferred embodiment, the composition comprises a reactive moisture-crosslinking hotmelt adhesive which has a low content of monomeric diisocyanates.

In a further aspect, the invention relates to a process for reducing the content of monomeric diisocyanates as claimed in claim 27. This process allows, in an elegant manner, a very great problem in polyurethane chemistry, specifically the presence of fractions of undesired diisocyanate monomers, to be solved inexpensively and efficiently, with the advantage that the monomers are not simply depleted but are incorporated in a useful manner.

Particularly preferred embodiments of the invention are the subject matter of the dependent claims.

Ways of Performing the Invention

In a first aspect of the invention, the present invention provides compounds VB of the formula (I) having isocyanate groups and aldimino groups.

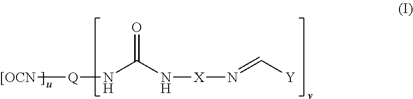

In this formula, Q is the radical of a polyisocyanate having (u+v) terminal isocyanate groups after removal of all isocyanate groups. In addition, u is 1 or 2 and v is 1 or 2.

Y is the radical of the formula (I a) or (I b).

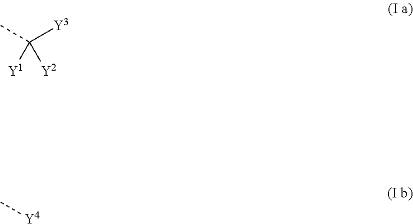

In these formulae, Y¹ and Y² are either
each independently a monovalent hydrocarbon radical having 1 to 12 carbon atoms,
or together are a divalent hydrocarbon radical which has 4 to 20 carbon atoms and is part of an optionally substituted, carbocyclic ring having 5 to 8, preferably 6, carbon atoms.

Y³ is a monovalent hydrocarbon radical which optionally has at least one heteroatom, especially oxygen in the form of ether, carbonyl or ester groups.

Y⁴
is a substituted or unsubstituted aryl or heteroaryl group which has a ring size of 5 to 8, preferably 6, atoms, or is

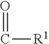

where R¹ is a hydrogen atom or an alkoxy group, or is a substituted or unsubstituted alkenyl or arylalkenyl group having at least 6 carbon atoms.

Finally, X is the radical of a diamine DA with two primary amino groups after the removal of these two amino groups. However, the proviso applies here that at least one of the two primary amino groups of the diamine DA is an aliphatic amino group and the two primary amino groups of the diamine DA differ from one another either
in the number of hydrogen atoms on the carbon atoms (C_{α}) in the α position to the particular amino group by at least one

or
in the number of hydrogen atoms on the carbon atoms (C_{β}) in the β position to the particular amino group by at least two.

In the present document, substance names beginning with “poly”, such as polyisocyanate, polyamine, polyol or polyaldimine, denote substances which, in a formal sense, contain two or more of the functional groups which occur in their name per molecule.

In the present document, the term “primary amino group” denotes an NH₂ group which is bonded to an organic radical, while the term “secondary amino group” denotes an NH group which is bonded to two organic radicals which may also together be part of a ring.

In the present document, “aliphatic amino group” denotes an amino group which is bonded to an aliphatic, cycloaliphatic or arylaliphatic radical. It thus differs from an “aromatic amino group”, which is bonded directly to an aromatic or heteroaromatic radical, as, for example, in aniline or 2-aminopyridine.

In the present document, the term “polymer” firstly embraces a collective of macromolecules which are chemically homogeneous but different in relation to degree of polymerization, molar mass and chain length, which has been prepared by a poly reaction (polymerization, polyaddition, polycondensation). The term secondly also embraces derivatives of such a collective of macromolecules from poly reactions, i.e. compounds which have been obtained by reactions, for example additions or substitutions, of functional groups on given macromolecules, and which may be chemically homogeneous or chemically inhomogeneous. The term further also comprises what are known as prepolymers, i.e. reactive oligomeric preliminary adducts whose functional groups are involved in the formation of macromolecules.

The term “polyurethane polymer” embraces all polymers prepared by what is known as the diisocyanate polyaddition process. This also includes those polymers which are virtually or entirely free of urethane groups. Examples of polyurethane polymers are polyetherpolyurethanes, polyesterpolyurethanes, polyetherpolyureas, polyureas, polyesterpolyureas, polyisocyanurates and polycarbodiimides.

The dotted lines in the formulae in this document each represent the bond between a substituent and the corresponding molecular radical.

“Room temperature” denotes a temperature of 25° C.

Y is preferably the radical of the formula (I a).

Y¹ and Y² are preferably each a methyl group.

Y³ is a monovalent hydrocarbon radical which optionally has at least one heteroatom, especially oxygen in the form of ether, carbonyl or ester groups.

Firstly, Y³ may especially be a branched or unbranched alkyl, cycloalkyl, alkylene or cycloalkylene group which optionally has at least one heteroatom, especially ether oxygen.

Secondly, Y³ may especially be a substituted or unsubstituted aryl or arylalkyl group.

In addition, Y³ may especially be a radical of the formula O—R² or

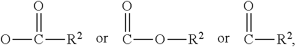

where R² in turn is an aryl, arylalkyl or alkyl group and is in each case substituted or unsubstituted.

Y³ is preferably a radical of the formula (II) or (III)

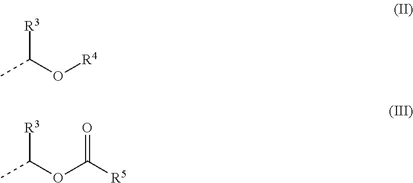

where R³ is a hydrogen atom or an alkyl or arylalkyl group, especially having 1 to 12 carbon atoms, preferably a hydrogen atom;

R⁴ is a hydrocarbon radical which has 1 to 30, especially 11 to 30, carbon atoms and optionally contains heteroatoms; and

R⁵
is a hydrogen atom,

or
is a linear or branched alkyl radical having 1 to 30, especially 11 to 30, carbon atoms, optionally with cyclic components and optionally with at least one heteroatom,

or
is a mono- or polyunsaturated, linear or branched hydrocarbon radical having 5 to 30 carbon atoms,

or
is an optionally substituted aromatic or heteroaromatic 5- or 6-membered ring.

Y³ is more preferably a radical of the formula (III).

The sum of u+v is preferably a value of 2 or 3.

A compound VB of the formula (I) having isocyanate groups and aldimino groups is obtainable by a process of reacting at least one dialdimine A of the formula (IV a) or (IV b) with at least one polyisocyanate of the formula (V) in the presence of a substoichiometric amount of water.

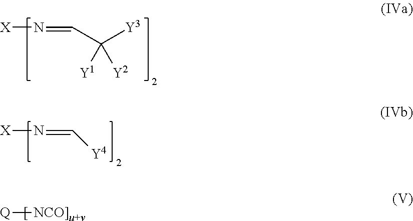

In the formulae (IV a), (IV b) and (V), X, Y¹, Y², Y³, Y⁴, u, v and Q are each as already defined.

The dialdimine A of the formula (IV a) or (IV b) is obtainable by a condensation reaction with elimination of water between a diamine DA of the formula (VI) and an aldehyde ALD of the formula (VII a) or (VII b). The aldehyde ALD of the formula (VII a) or (VII b) is used here in a stoichiometric amount or in a stoichiometric excess in relation to the amino groups of the diamine DA.

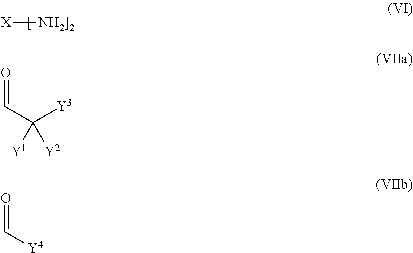

In the formulae (VI), (VII a) and (VII b), X, Y¹, Y², Y³ and Y⁴ are each as already defined.

It is essential for the present invention that the two primary amino groups of the diamine DA differ from one another either in the number of hydrogen atoms on the carbon atoms (C_{α}) in the α position (=1 position) to the particular amino group by at least one or in the number of hydrogen atoms on the carbon atoms (C_{β}) in the β position (=2 position) to the particular amino group by at least two.

H₂N—C_{α}—C_{β}—C_{γ}—C_{δ}—

The diamine DA thus has different substitution patterns on the α carbon atoms and on the β carbon atoms to the particular amino group. Diamines having such different substitution or dialdimines derived therefrom are also referred to as “asymmetric” in the present document. This different substitution leads to different reactivity of the two primary amino groups, especially toward isocyanate groups.

In one embodiment, the diamine DA thus differs in the substitution pattern on the carbon atoms which are in the α position to the primary amino groups.

Such diamines DA are, for example, 1,2-propanediamine, 2-methyl-1,2-propanediamine, 1,3-butanediamine, 1,3-diaminopentane (DAMP), 4-aminoethylaniline, 4-aminomethylaniline, 4-[(4-aminocyclohexyl)methyl]-aniline, 2-aminoethylaniline, 2-aminomethylaniline, 2-[(4-aminocyclohexyl)-methyl]aniline and 4-[(2-aminocyclohexyl)methyl]aniline.

In another embodiment, the diamine DA thus differs in the substitution pattern on the carbon atoms which are in the β position to the primary amino groups.

Such diamines DA are, for example, 2,2,4-trimethylhexamethylene-diamine (TMD), 1,5-diamino-2-butyl-2-ethylpentane, 1-amino-3-aminomethyl-3,5,5-trimethylcyclohexane (=isophoronediamine=IPDA) and 1,4-diamino-2,2,6-trimethylcyclohexane (TMCDA).

The diamine DA has two primary amino groups, at least one of which is aliphatic. The second amino group may be an aliphatic or aromatic amino group.

Not considered to be diamines DA of the formula (VI) are diamines whose amino groups differ from one another merely by one hydrogen atom on the carbon atoms (C_{β}) in the β position to the particular amino group. One example of such a diamine which is not a diamine DA is 2-methylpenta-methylenediamine (=1,5-diamino-2-methylpentane=MPMD). Likewise not considered to be diamines DA of the formula (VI) are diamines whose amino groups differ from one another only in the number of hydrogen atoms on the carbon atoms (C_{γ} and C_{δ}) in the γ and δ positions to the particular amino group. In all these cases, the different substitution pattern on the diamine brings about an only insignificant difference, if any, in reactivity of the amino groups, especially toward isocyanate groups.

The diamine DA of the formula (VI) is preferably selected from the group consisting of 1,3-diaminopentane (DAMP), 1,5-diamino-2-butyl-2-ethyl-pentane, 2,2,4-trimethylhexamethylenediamine (TMD) and 1-amino-3-amino-methyl-3,5,5-trimethylcyclohexane (=isophoronediamine=IPDA).

The diamines DA of the formula (VI) have two primary amino groups. Apart from these two amino groups, the diamines DA are free of moieties which are reactive with isocyanate groups; more particularly, they have no hydroxyl groups, no secondary amino groups and no mercapto groups.

To prepare a dialdimine A of the formula (IV a) or (IV b), aldehydes ALD of the formula (VII a) or (VII b) are used. The aldehydes ALD have the property that their Y¹, Y², Y³ and Y⁴ radicals have no moieties which are reactive with isocyanate groups; more particularly, Y¹, Y², Y³ and Y⁴ have no hydroxyl groups, no primary or secondary amino groups and no mercapto groups.

For preparation of a dialdimine A of the formula (IV a), suitable aldehydes ALD of the formula (VII a) are those where Y¹, Y² and Y³ are each as already defined.

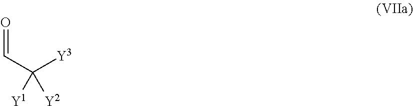

Aldehydes ALD of the formula (VII a) are tertiary aliphatic or tertiary cycloaliphatic aldehydes. Suitable aldehydes ALD of the formula (VII a) are, for example, pivalaldehyde (=2,2-dimethylpropanal), 2,2-dimethylbutanal, 2,2-diethylbutanal, 1-methylcyclopentanecarboxaldehyde, 1-methylcyclohexanecarboxaldehyde; ethers formed from 2-hydroxy-2-methylpropanal and alcohols such as propanol, isopropanol, butanol and 2-ethylhexanol; esters formed from 2-formyl-2-methylpropionic acid or 3-formyl-3-methylbutyric acid and alcohols such as propanol, isopropanol, butanol and 2-ethylhexanol; esters formed from 2-hydroxy-2-methylpropanal and carboxylic acids such as butyric acid, isobutyric acid and 2-ethylhexanoic acid; and the ethers and esters, described hereinafter as particularly suitable, of 2,2-disubstituted 3-hydroxypropanals, 3-hydroxybutanals or analogous higher aldehydes, especially of 2,2-dimethyl-3-hydroxypropanal.

Particularly suitable aldehydes ALD of the formula (VII a) are firstly aldehydes ALD1 of the formula (VIII), i.e. aldehydes ALD of the formula (VII a) with the Y³ radical of the formula (II).

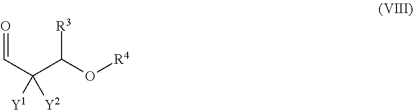

In the formula (VIII), Y¹, Y², R³ and R⁴ are each as already defined.

In formula (VIII), Y¹ and Y² are preferably each a methyl group, and R³ is preferably a hydrogen atom.

Aldehydes ALD1 of the formula (VIII) are ethers of aliphatic, cycloaliphatic or arylaliphatic 2,2-disubstituted 3-hydroxyaldehydes with alcohols or phenols of the formula R⁴—OH, for example fatty alcohols or a phenol. Suitable 2,2-disubstituted 3-hydroxyaldehydes are in turn obtainable from aldol reactions, especially crossed aldol reactions, between primary or secondary aliphatic aldehydes, especially formaldehyde, and secondary aliphatic, secondary cycloaliphatic or secondary arylaliphatic aldehydes, for example isobutyraldehyde, 2-methylbutyraldehyde, 2-ethylbutyraldehyde, 2-methylvaleraldehyde, 2-ethylcapronaldehyde, cyclopentanecarboxaldehyde, cyclohexanecarboxaldehyde, 1,2,3,6-tetrahydrobenzaldehyde, 2-methyl-3-phenylpropionaldehyde, 2-phenylpropionaldehyde (hydratropaldehyde) or diphenylacetaldehyde.

Examples of such aldehydes ALD1 of the formula (VIII) are 2,2-dimethyl-3-phenoxypropanal, 3-cyclohexyloxy-2,2-dimethylpropanal, 2,2-dimethyl-3-(2-ethylhexyloxy)propanal, 2,2-dimethyl-3-lauroxypropanal and 2,2-dimethyl-3-stearoxypropanal.

Particularly suitable aldehydes ALD of the formula (VII a) are secondly aldehydes ALD2 of the formula (IX), i.e. aldehydes ALD of the formula (VII a) with the Y³ radical of the formula (III).

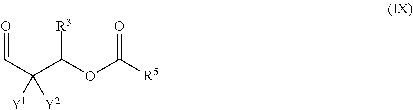

In formula (IX), Y¹, Y², R³ and R⁵ are each as already defined.

In formula (IX), Y¹ and Y² are preferably each a methyl group, and R³ is preferably a hydrogen atom.

Aldehydes ALD2 of the formula (IX) are esters of the 2,2-disubstituted 3-hydroxyaldehydes already described, for example 2,2-dimethyl-3-hydroxypropanal, 2-hydroxymethyl-2-methylbutanal, 2-hydroxymethyl-2-ethylbutanal, 2-hydroxymethyl-2-methylpentanal, 2-hydroxymethyl-2-ethylhexanal, 1-hydroxymethylcyclopentanecarboxaldehyde, 1-hydroxymethylcyclo hexane-carboxaldehyde, 1-hydroxymethylcyclohex-3-enecarboxaldehyde, 2-hydroxy-methyl-2-methyl-3-phenylpropanal, 3-hydroxy-2-methyl-2-phenylpropanal and 3-hydroxy-2,2-diphenylpropanal, with suitable carboxylic acids.

Examples of suitable carboxylic acids are firstly aliphatic carboxylic acids, such as formic acid, acetic acid, propionic acid, butyric acid, isobutyric acid, valeric acid, caproic acid, 2-ethylcaproic acid, capric acid, lauric acid, tridecanoic acid, myristic acid, pentadecanoic acid, palmitic acid, margaric acid, stearic acid, nonadecanoic acid, arachic acid, palmitoleic acid, oleic acid, erucic acid, linoleic acid, linolenic acid, eleostearic acid, arachidonic acid, fatty acids from the industrial hydrolysis of natural oils and fats, for example rapeseed oil, sunflower oil, linseed oil, olive oil, coconut oil, oil palm kernel oil and oil palm oil, and also technical grade mixtures of fatty acids which comprise such acids. Suitable carboxylic acids are secondly aromatic carboxylic acids, for example benzoic acid or the positionally isomeric toluic acids, ethyl- or isopropyl- or tert-butyl- or methoxy- or nitrobenzoic acids.

Preferred aldehydes ALD2 of the formula (IX) are 3-benzoyloxy-2,2-dimethylpropanal, 3-cyclohexanoyloxy-2,2-dimethylpropanal, 2,2-dimethyl-3-(2-ethylhexyloxy)propanal, 2,2-dimethyl-3-lauroyloxypropanal, 2,2-dimethyl-3-myristoyloxypropanal, 2,2-dimethyl-3-palmitoyloxypropanal, 2,2-dimethyl-3-stearoyloxypropanal, and analogous esters of other 2,2-disubstituted 3-hydroxyaldehydes.

In a particularly preferred embodiment, R⁵ is selected from the group consisting of phenyl, cyclohexyl and the C₁₁-, C₁₃-, C₁₅- and C₁₇-alkyl groups.

A particularly preferred aldehyde ALD2 of the formula (IX) is 2,2-dimethyl-3-lauroyloxypropanal.

In a preferred preparation method of the aldehyde ALD2 of the formula (IX), a 2,2-disubstituted 3-hydroxyaldehyde, for example 2,2-dimethyl-3-hydroxypropanal, which can be prepared, for example, from formaldehyde (or paraformaldehyde) and isobutyraldehyde, optionally in situ, is reacted with a carboxylic acid to give the corresponding ester. This esterification can be effected without the use of solvents by known methods, described, for example, in Houben-Weyl, “Methoden der organischen Chemie” [Methods of Organic Chemistry], vol. VIII, pages 516-528.

In a particularly preferred embodiment, the aldehyde ALD of the formula (VII a) is odorless. An “odorless” substance is understood to mean a substance which has such a low odor that most humans cannot smell it, i.e. is imperceptible with the nose.

Odorless aldehydes ALD of the formula (VII a) are firstly especially aldehydes ALD1 of the formula (VIII) in which the R⁴ radical is a hydrocarbon radical which has 11 to 30 carbon atoms and optionally contains heteroatoms.

Secondly, odorless aldehydes ALD of the formula (VII a) are especially aldehydes ALD2 of the formula (IX) in which the R⁵ radical is either a linear or branched alkyl group having 11 to 30 carbon atoms, optionally having cyclic components, and optionally having at least one heteroatom, especially having at least one ether oxygen, or is a mono- or polyunsaturated linear or branched hydrocarbon chain having 11 to 30 carbon atoms.

Examples of odorless aldehydes ALD2 of the formula (IX) are esterification products of the 2,2-disubstituted 3-hydroxyaldehydes already mentioned with carboxylic acids, for example lauric acid, tridecanoic acid, myristic acid, pentadecanoic acid, palmitic acid, margaric acid, stearic acid, nonadecanoic acid, arachic acid, palmitoleic acid, oleic acid, erucic acid, linoleic acid, linolenic acid, eleostearic acid, arachidonic acid, fatty acids from the industrial hydrolysis of natural oils and fats, for example rapeseed oil, sunflower oil, linseed oil, olive oil, coconut oil, oil palm kernel oil and oil palm oil, and technical grade mixtures of fatty acids which comprise these acids. Preferred aldehydes of the formula (IX) are 2,2-dimethyl-3-lauroyloxypropanal, 2,2-dimethyl-3-myristoyloxypropanal, 2,2-dimethyl-3-palmitoyloxypropanal and 2,2-dimethyl-3-stearoyloxypropanal. Particular preference is given to 2,2-dimethyl-3-lauroyloxypropanal.

For preparation of a dialdimine A of the formula (IV b), aldehydes ALD of the formula (VII b) are suitable.

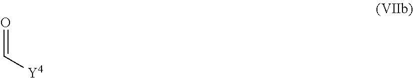

Suitable aldehydes ALD of the formula (VII b) are aromatic aldehydes, for example benzaldehyde, 2- and 3- and 4-tolualdehyde, 4-ethyl- and 4-propyl- and 4-isopropyl- and 4-butylbenzaldehyde, 2,4-dimethylbenzaldehyde, 2,4,5-trimethylbenzaldehyde, 4-acetoxybenzaldehyde, 4-anisaldehyde, 4-ethoxybenzaldehyde, the isomeric di- and trialkoxybenzaldehydes, 2-, 3- and 4-nitrobenzaldehyde, 2- and 3- and 4-formylpyridine, 2-furfuraldehyde, 2-thio-phenecarbaldehyde, 1- and 2-naphthylaldehyde, 3- and 4-phenyloxybenzaldehyde; quinoline-2-carbaldehyde and the 3, 4, 5, 6, 7 and 8 positional isomers thereof, and anthracene-9-carbaldehyde.

Suitable aldehydes ALD of the formula (VII b) are additionally glyoxal, glyoxalic esters, for example methyl glyoxalate, cinamaldehyde and substituted cinamaldehydes.

The dialdimine A of the formula (IV a) with aliphatic aldimino groups and the dialdimine A of the formula (IV b) with aromatic aldimino groups have the property that their aldimino groups cannot tautomerize to enamino groups, since they do not contain any hydrogen in the α position to the carbon atom of the aldimino group.

Dialdimines A which are prepared proceeding from odorless aldehydes of the particularly preferred embodiment described above are odorless. Such odorless dialdimines A are particularly preferred for reaction with polyisocyanates, since odorless compounds VB are obtainable in this way.

Preferred dialdimines A are those which have the formula (IV a).

In a preferred embodiment, a polyisocyanate of the formula (V) suitable for reaction with at least one dialdimine A is a polyurethane polymer PUP having isocyanate groups.

A suitable polyurethane polymer PUP having isocyanate groups is obtainable by the reaction of at least one polyol with at least one polyisocyanate.

The polyols used for the preparation of a polyurethane polymer PUP may, for example, be the following commercially available polyols or mixtures thereof:

polyetherpolyols, also known as polyoxyalkylenepolyols or oligoetherols, which are polymerization products of ethylene oxide, 1,2-propylene oxide, 1,2- or 2,3-butylene oxide, oxetane, tetrahydrofuran or mixtures thereof, possibly polymerized with the aid of a starter molecule with two or more active hydrogen atoms, for example water, ammonia or compounds having a plurality of OH or NH groups, for example 1,2-ethanediol, 1,2- and 1,3-propanediol, neopentyl glycol, diethylene glycol, triethylene glycol, the isomeric dipropylene glycols and tripropylene glycols, the isomeric butanediols, pentanediols, hexanediols, heptanediols, octanediols, nonanediols, decanediols, undecanediols, 1,3- and 1,4-cyclohexane-dimethanol, bisphenol A, hydrogenated bisphenol A, 1,1,1-trimethylolethane, 1,1,1-trimethylolpropane, glycerol, aniline, and mixtures of the aforementioned compounds. It is possible to use either polyoxyalkylenepolyols which have a low degree of unsaturation (measured to ASTM D-2849-69 and reported in milliequivalents of unsaturation per gram of polyol (meq/g)), prepared, for example, with the aid of double metal cyanide complex catalysts (DMC catalysts), or polyoxyalkylenepolyols with a higher degree of unsaturation, prepared, for example, with the aid of anionic catalysts such as NaOH, KOH, CsOH or alkali metal alkoxides.

Particularly suitable polyetherpolyols are polyoxyalkylenediols and -triols, especially polyoxyalkylenediols. Particularly suitable polyoxyalkylene-di- and -triols are polyoxyethylenedi- and -triols and polyoxypropylenedi- and -triols.

Particularly suitable polyoxypropylenediols and -triols have a degree of unsaturation lower than 0.02 meq/g and a molecular weight in the range from 1000 to 30 000 g/mol, and also polyoxypropylenediols and -triols with a molecular weight of 400 to 8000 g/mol. In the present document, “molecular weight” or “molar mass” is always understood to mean the molecular weight average Mₙ. Especially suitable are polyoxypropylenediols with a degree of unsaturation less than 0.02 meq/g and a molecular weight in the range from 1000 to 12 000 and especially between 1000 and 8000 g/mol. Such polyetherpolyols are sold, for example, under the trade name Acclaim® by Bayer.

Likewise particularly suitable are so-called “EO-endcapped” (ethylene oxide-endcapped) polyoxypropylenediols and -triols. The latter are specific polyoxypropylenepolyoxyethylenepolyols which are obtained, for example, by alkoxylating pure polyoxypropylenepolyols with ethylene oxide on completion of the polypropoxylation, and have primary hydroxyl groups as a result.

Styrene-acrylonitrile- or acrylonitrile-methyl methacrylate-grafted polyether-polyols.

Polyesterpolyols, also known as oligoesterols, prepared by known processes, especially the polycondensation of hydroxycarboxylic acids or the polycondensation of aliphatic and/or aromatic polycarboxylic acids with di- or polyhydric alcohols.

Especially suitable polyesterpolyols are those prepared from di- to trihydric, especially dihydric, alcohols, for example ethylene glycol, diethylene glycol, propylene glycol, dipropylene glycol, neopentyl glycol, 1,4-butanediol, 1,5-pentanediol, 3-methyl-1,5-hexanediol, 1,6-hexanediol, 1,8-octanediol, 1,10-decanediol, 1,12-dodecanediol, 1,12-hydroxystearyl alcohol, 1,4-cyclohexanedimethanol, dimer fatty acid diol (dimer diol), neopentyl glycol hydroxypivalate, glycerol, 1,1,1-trimethylolpropane or mixtures of the aforementioned alcohols, with organic di- or tricarboxylic acids, especially dicarboxylic acids, or the anhydrides or esters thereof, for example succinic acid, glutaric acid, adipic acid, trimethyladipic acid, suberic acid, azelaic acid, sebacic acid, dodecanedicarboxylic acid, maleic acid, fumaric acid, dimer fatty acid, phthalic acid, phthalic anhydride, isophthalic acid, terephthalic acid, dimethyl terephthalate, hexahydrophthalic acid, trimellitic acid and trimellitic anhydride, or mixtures of the aforementioned acids, and also polyesterpolyols formed from lactones, for example from ε-caprolactone, and starters such as the aforementioned di- or trihydric alcohols.

Particularly suitable polyesterpolyols are polyesterdiols. Especially suitable polyesterdiols are those prepared from adipic acid, azelaic acid, sebacic acid, dodecanedicarboxylic acid, dimer fatty acid, phthalic acid, isophthalic acid and terephthalic acid as the dicarboxylic acid, and from ethylene glycol, diethylene glycol, neopentyl glycol, 1,4-butanediol, 1,6-hexanediol, dimer fatty acid diol and 1,4-cyclohexanedimethanol as the dihydric alcohol. Also especially suitable are polyesterdiols prepared from ε-caprolactone and one of the aforementioned dihydric alcohols as the starter.

Especially suitable substances are room temperature liquid, amorphous, partly crystalline and crystalline polyesterdi- and -triols, especially polyesterdiols. Suitable room temperature liquid polyesterpolyols are solid not far below room temperature, for example at temperatures between 0° C. and 25° C., and are preferably used in combination with at least one amorphous, partly crystalline or crystalline polyesterpolyol.

Polycarbonatepolyols, as obtainable by reaction, for example, of the abovementioned alcohols—used to form the polyesterpolyols—with dialkyl carbonates such as dimethyl carbonate, diaryl carbonates such as diphenyl carbonate, or phosgene.

Particularly suitable substances are polycarbonatediols, especially amorphous polycarbonatediols.

Likewise suitable as polyols are block copolymers which bear at least two hydroxyl groups and have at least two different blocks with polyether, polyester and/or polycarbonate structure of the type described above.

Polyacrylate- and polymethacrylatepolyols.

Poly-hydroxy-functional fats and oils, for example natural fats and oils, especially castor oil; or polyols—known as oleochemical polyols—obtained by chemical modification of natural fats and oils, for example the epoxy polyesters or epoxy polyethers obtained by epoxidation of unsaturated oils and subsequent ring opening with carboxylic acids or alcohols, or polyols obtained by hydroformylation and hydrogenation of unsaturated oils; or polyols obtained from natural fats and oils by degradation processes such as alcoholysis or ozonolysis and subsequent chemical linkage, for example by transesterification or dimerization, of the degradation products or derivatives thereof thus obtained. Suitable degradation products of natural fats and oils are especially fatty acids and fatty alcohols, and also fatty acid esters, especially the methyl esters (FAME), which can be derivatized, for example, by hydroformylation and hydrogenation to hydroxy fatty acid esters.

Polyhydrocarbonpolyols, also known as oligohydrocarbonols, for example poly-hydroxy-functional ethylene-propylene, ethylene-butylene or ethylene-propylene-diene copolymers, as produced, for example, by Kraton Polymers, or poly-hydroxy-functional copolymers of dienes such as 1,3-butadiene or diene mixtures, and vinyl monomers such as styrene, acrylonitrile or isobutylene, or poly-hydroxy-functional polybutadienepolyols, for example those which are prepared by copolymerization of 1,3-butadiene and allyl alcohol and may also be hydrogenated.

Poly-hydroxy-functional acrylonitrile/butadiene copolymers, as can be prepared, for example, from epoxides or amino alcohols and carboxyl-terminated acrylonitrile/butadiene copolymers (commercially available under the Hycar® CTBN name from Hanse Chemie).

These polyols mentioned preferably have a mean molecular weight of 250-30 000 g/mol, especially of 400-20 000 g/mol, and preferably have a mean OH functionality in the range from 1.6 to 3.

In addition to these polyols mentioned, small amounts of low molecular weight di- or polyhydric alcohols, for example 1,2-ethanediol, 1,2- and 1,3-propanediol, neopentyl glycol, diethylene glycol, triethylene glycol, the isomeric dipropylene glycols and tripropylene glycols, the isomeric butanediols, pentanediols, hexanediols, heptanediols, octanediols, nonanediols, decanediols, undecanediols, 1,3- and 1,4-cyclohexanedimethanol, hydrogenated bisphenol A, dimeric fatty alcohols, 1,1,1-trimethylolethane, 1,1,1-tri-methylolpropane, glycerol, pentaerythritol, low molecular weight alkoxylation products of the aforementioned di- and polyhydric alcohols, and mixtures of the aforementioned alcohols, can be used additionally in the preparation of a polyurethane polymer PUP.

The polyisocyanates used for the preparation of a polyurethane polymer PUP may be commercial aliphatic, cycloaliphatic or aromatic polyisocyanates, especially diisocyanates, for example the following:

1,6-hexamethylene diisocyanate (HDI), 2-methylpentamethylene 1,5-diisocyanate, 2,2,4- and 2,4,4-trimethyl-1,6-hexamethylene diisocyanate (TMDI), 1,10-decamethylene diisocyanate, 1,12-dodecamethylene diiso-cyanate, lysine diisocyanate and lysine ester diisocyanate, cyclohexane 1,3-and 1,4-diisocyanate and any desired mixtures of these isomers, 1-methyl-2,4-and -2,6-diisocyanatocyclohexane and any desired mixtures of these isomers (HTDI or H₆TDI), 1-isocyanato-3,3,5-trimethyl-5-isocyanatomethylcyclohexane (=isophorone diisocyanate or IPDI), perhydro-2,4′- and -4,4′-diphenylmethane diisocyanate (HMDI or H₁₂MDI), 1,4-diisocyanato-2,2,6-trimethylcyclohexane (TMCDI), 1,3- and 1,4-bis(isocyanatomethyl)cyclohexane, m- and p-xylylene diisocyanate (m- and p-XDI), m- and p-tetramethyl-1,3- and -1,4-xylylene diisocyanate (m- and p-TMXDI), bis(1-isocyanato-1-methylethyl)naphthalene, 2,4- and 2,6-tolylene diisocyanate and any desired mixtures of these isomers (TDI), 4,4′-, 2,4′- and 2,2′-diphenylmethane diisocyanate and any desired mixtures of these isomers (MDI), 1,3- and 1,4-phenylene diisocyanate, 2,3,5,6-tetramethyl-1,4-diisocyanatobenzene, naphthalene 1,5-diisocyanate (NDI), 3,3′-dimethyl-4,4′-diisocyanatodiphenyl (TODD, dianisidine diisocyanate (DADI), oligomers and polymers of the aforementioned isocyanates, and any desired mixtures of the aforementioned isocyanates. Preference is given to monomeric diisocyanates, especially MDI, TDI, HDI and IPDI.

The polyurethane polymer PUP is prepared in a known manner directly from the polyisocyanates and the polyols, or by stepwise adduction processes, as also known as chain extension reactions.

In a preferred embodiment, the polyurethane polymer PUP is prepared via a reaction of at least one polyisocyanate and at least one polyol, the isocyanate groups being present in a stoichiometric excess relative to the hydroxyl groups. The ratio between isocyanate and hydroxyl groups is advantageously 1.3 to 5, especially 1.5 to 3.

The polyurethane polymer PUP has a molecular weight of preferably more than 500 g/mol, especially one between 1000 and 50 000 g/mol, preferably one between 2000 and 30 000 g/mol.

Moreover, the polyurethane polymer PUP preferably has a mean functionality in the range from 1.8 to 3.

In a preferred embodiment, the polyurethane polymer PUP is a room temperature solid polyurethane polymer PUP1. A room temperature solid polyurethane polymer PUP1 is advantageously obtainable proceeding from polyetherpolyols, polyesterpolyols and polycarbonatepolyols. Especially suitable substances are room temperature liquid, amorphous, partly crystalline and crystalline polyester- and polycarbonatedi- and -triols, especially polyesterdiols and polycarbonatediols, though room temperature liquid polyester- and polycarbonatedi- and -triols are solid not far below room temperature, for example at temperatures between 0° C. and 25° C., and are preferably used in combination with at least one amorphous, partly crystalline or crystalline polyol.

The polyester- and polycarbonatedi- and -triols advantageously have a molecular weight of 500 to 5000 g/mol.

A room temperature solid polyurethane polymer PUP1 may be crystalline, partly crystalline or amorphous. A partly crystalline or amorphous polyurethane polymer PUP1 has only limited free flow, if any, at room temperature, which means more particularly that it has a viscosity of more than 5000 Pa·s at 20° C.

The polyurethane polymer PUP1 preferably has a mean molecular weight of 1000 to 10 000 g/mol, especially of 2000 to 5000 g/mol.

In a further embodiment, a polyisocyanate of the formula (V) suitable for reaction with at least one dialdimine A is an oligomeric polyisocyanate PI which is prepared from an aliphatic, cycloaliphatic or aromatic diisocyanate.

Suitable polyisocyanates PI are oligomers or oligomer mixtures of diisocyanates, especially of HDI, IPDI, TDI and MDI. Commercially available types are especially HDI biurets, for example as Desmodur® N 100 and N 3200 (Bayer), Tolonate® HDB and HDB-LV (Rhodia) and Duranate® 24A-100 (Asahi Kasei); HDI isocyanurates, for example as Desmodur® N 3300, N 3600 and N 3790 BA (all from Bayer), Tolonate® HDT, HDT-LV and HDT-LV2 (Rhodia), Duranate® TPA-100 and THA-100 (Asahi Kasei) and Coronate® HX (Nippon Polyurethane); HDI uretdiones, for example as Desmodur® N 3400 (Bayer); HDI iminooxadiazinediones, for example as Desmodur® XP 2410 (Bayer); HDI allophanates, for example as Desmodur® VP LS 2102 (Bayer); IPDI isocyanurates, for example in solution as Desmodur® Z 4470 (Bayer) or in solid form as Vestanat® T1890/100 (Degussa); TDI oligomers, for example as Desmodur® IL (Bayer); mixed isocyanurates based on TDI/HDI, for example as Desmodur® HL (Bayer).

Preferred polyisocyanates PI are the oligomers of HDI and/or IPDI, especially the isocyanurates.

The aforementioned polyisocyanates PI are typically mixtures of substances with different degrees of oligomerization and/or chemical structures. They preferably have a mean NCO functionality of 2.1 to 4.0 and contain especially isocyanurate, iminooxadiazinedione, uretdione, urethane, biuret, allophanate, carbodiimide, uretonimine or oxadiazinetrione groups.

It is likewise possible that a polyisocyanate of the formula (V) suitable for reaction with at least one dialdimine A is a mixture consisting of at least one polyurethane polymer PUP and at least one polyisocyanate Pl.

The polyisocyanate of the formula (V) suitable for reaction with at least one dialdehyde A is preferably a polyurethane polymer PUP having isocyanate groups. The polyisocyanate of the formula (V) is more preferably a room temperature solid polyurethane polymer PUP1.

In the reaction of at least one dialdimine A of the formula (IV a) or (IV b) with at least one polyisocyanate of the formula (V) in the presence of a substoichiometric amount of water to prepare a compound VB of the formula (I) having isocyanate groups and aldimino groups, the substoichiometric amount of water is preferably not more than 0.5 mol, especially not more than 0.3 mol, of water per equivalent of aldimino groups. This reaction can optionally be effected in the presence of a catalyst, for example of an acid, especially of a carboxylic acid.

The dialdimines A have the property that they are storage-stable under suitable conditions, especially with exclusion of moisture. On ingress of moisture, their aldimino groups can be hydrolyzed in a formal sense to amino groups via intermediates to release the corresponding aldehyde used to prepare the aldimine. Since this hydrolysis reaction is reversible and the chemical equilibrium is clearly toward the aldimine side, it can be assumed that, in the absence of groups reactive toward amines, only some of the aldimino groups are hydrolyzed. In the presence of isocyanate groups, the hydrolysis equilibrium shifts, since the aldimino groups being hydrolyzed react irreversibly with the isocyanate groups to give urea groups. The reaction of the isocyanate groups with the aldimino groups being hydrolyzed need not necessarily proceed via free amino groups. Reactions with intermediates of the hydrolysis reaction are also possible. For example, it is conceivable that an aldimino group being hydrolyzed reacts directly in the form of a hemiaminal with an isocyanate group. When a dialdimine A is contacted with a polyisocyanate of the formula (V) in the presence of a substoichiometric amount of water, this forms compounds containing aldimino groups. The two aldimino groups of the dialdimine A of the formula (IV a) or (IV b) have a significant difference in reactivity; as a result, the more reactive aldimino group reacts preferentially with hydrolysis with the isocyanate groups, whereas the less reactive aldimino group is substantially preserved. In this way, the above-described compounds VB of the formula (I) are formed with some degree of selectivity. The greater the difference in reactivity between the aldimino groups of the dialdimine A, the more selectively the compound VB is formed.

As described, the dialdimines A of the formula (IV a) or (IV b) are based on asymmetric diamines DA of the formula (VI) and aldehydes ALD of the formula (VII a) or (VII b). The different reactivity of the two amino groups in a diamine DA, which is due to the different substitution, is transferred directly to a dialdimine of this diamine DA, in which the two aldimino groups likewise have a different reactivity. In the special case of the dialdimines A, the difference in reactivity in the aldimino groups, caused by the sterically demanding structure of the parent aldehyde ALD of the aldimino group, is probably actually enhanced by virtue of the sterically demanding aldehyde radical additionally limiting the accessibility of the aldimino groups—especially when they are present in semihydrolyzed form as hemiaminal groups—and thus lowering the reactivity of the slower aldimino group to a greater than proportional degree compared to that of the faster aldimino group.

It is not impossible that by-products also form in this reaction. These are, for example, compounds of the formula (X).

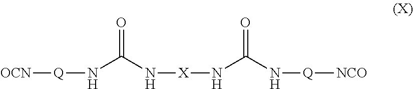

In the formula (X), Q and X are each as already defined.

Owing to the use of an asymmetric dialdimine A and of the asymmetric diamine DA, these compounds of the formula (X), however, form only in small amounts.

For the described reaction to prepare a compound VB of the formula (I) having isocyanate groups and aldimino groups, the polyisocyanate of the formula (V) is either first mixed with the dialdimine A of the formula (IV a) or (IV b) and this mixture is then admixed with the water, or the polyisocyanate is mixed directly with a previously prepared mixture of the dialdimine A and the water. What is crucial for the formation of the compound VB is the fact that the dialdimine A which undergoes partial hydrolysis reacts with available isocyanate groups of the polyisocyanate, the desired degree of reaction being controlled via the amount of water used. The polyisocyanate should be absolutely prevented from coming into contact with water unless the dialdimine A is present as a reactant. The reaction can be effected under conditions customary for reactions with isocyanate groups, optionally in the presence of at least one suitable catalyst. Preference is given to conducting the reaction at a temperature of 20 to 150° C. In the case that the polyisocyanate of the formula (V) is a room temperature solid polyurethane polymer PUP1, the reaction is preferably conducted at a temperature at which such a polyurethane polymer PUP1 is liquid.

In the reaction described, the ratio between the water and the aldimino groups of the dialdimine A is advantageously at most 0.5, especially 0.3 to 0.5, mol of water per equivalent of aldimino groups.

In the reaction described, the ratio between the number of isocyanate groups of the polyisocyanate of the formula (V) to the number of aldimino groups of the dialdimine A is advantageously at least 1, preferably 1 to 5.

After the reaction with the water, the ratio between the number of all aldimino groups present in the composition and the number of all isocyanate groups present in the composition is advantageously 0.1 to 1.1, preferably 0.2 to 1.0, more preferably 0.2 to 0.9.

Preparing the compound VB in the manner described via a dialdimine A appears to equate to an unnecessary diversion and is therefore not obvious. More obvious and apparently more advantageous would be the use of a diamine DA converted to the aldimine only on one side, i.e. an aminoaldimine, which is a monoaldimine with a primary amino group, since such a monoaldimine can be added directly onto the polyisocyanate.

On closer inspection of this supposedly more advantageous synthesis route, however, great weaknesses are revealed. Firstly, the desired monoaldimines are not easy to obtain in pure form because they are usually formed with low selectivity from the diamines and are obtained as mixtures with the dialdimines and unconverted diamines, or because they tend to further reactions, especially to cyclization, for example to form aminals, or to addition reactions, for example with amide formation, for instance when the monoaldimines have ester groups. Secondly, the monoaldimines react, owing to the extremely rapid reaction between primary amino groups and isocyanate groups, exceptionally vigorously with the polyisocyanate and lead, when mixed in, to inhomogeneous adducts of poor quality. The reaction route described above here using a dialdimine A to give compounds VB of the formula (I) avoids these difficulties in an elegant manner. Dialdimines A can be mixed homogeneously into a polyisocyanate.

The compounds VB of the formula (I) having isocyanate groups and aldimine groups have the property that their aldimino groups cannot tautomerize to enamino groups, since they do not contain hydrogen as a substituent in cc position to the carbon atom of the aldimino group. Owing to this property, they form, together with isocyanate groups, particularly storable, i.e. substantially viscosity-stable, mixtures, even if highly reactive aromatic isocyanate groups, such as those derived from TDI and MDI, are present in these mixtures.

The compound VB of the formula (I) reacts under the influence of moisture by virtue of the isocyanate groups reacting with the aldimino groups which hydrolyze as a result of the moisture, and the isocyanate groups with one another. In the case that the index u in the formula (I) is greater than or equal to the index v, the compound VB is self-curing by means of moisture. This means that the action of moisture can crosslink the compound VB even alone to give a high molecular weight polyurethane polymer. It is possible in this case to obtain a moisture-curing composition without a polyisocyanate P as detailed hereinafter.

The compound VB is storage-stable, i.e. it can be stored with exclusion of moisture in a suitable package or arrangement, for example a vat, a hobbock, a bucket, a bag or a cartridge, over a period of several months up to one year and longer, without its performance properties or its properties after curing changing to a degree relevant for the use thereof. Typically, the storage stability is determined via the measurement of the viscosity.

A further aspect of the present invention relates to a composition comprising

a) at least one of the above-described compounds VB of the formula (I) having isocyanate groups and aldimino groups, especially prepared by the above-described process, and

b) at least one polyisocyanate P.

In one embodiment, the polyisocyanate P is a polyurethane polymer PUP having isocyanate groups, as has already been described above for the compound VB of the formula (I), or in the preparation thereof. In a further embodiment, the polyisocyanate P is an oligomeric polyisocyanate PI, as has likewise already been described above for the compound VB of the formula (I), or in the preparation thereof. The polyisocyanate P is preferably a polyurethane polymer PUP having isocyanate groups. The polyisocyanate P present in the composition is more preferably the same polyisocyanate as the polyisocyanate having (u+v) terminal isocyanate groups, from which Q in the compound VB of the formula (I) is derived.

Such a composition is preferably obtainable by using at least one polyisocyanate of the formula (V) and at least one dialdimine A and water in such a ratio that, after the reaction, a residue of the polyisocyanate of the formula (V) remains in the composition as polyisocyanate P.

The composition described has a low content of monomeric diisocyanates.

It has been found that, completely surprisingly, the reaction of a polyisocyanate of the formula (V) which has a particular content of monomeric diisocyanate with a dialdimine A in the presence of a substoichiometric amount of water in the manner described above leads to a composition which has a surprisingly low content of monomeric diisocyanates. This is significantly lower than that of the polyisocyanate of the formula (V) before the reaction.

The surprisingly low content of monomeric diisocyanates is probably achieved by virtue of the fact that, in the described reaction of at least one polyisocyanate of the formula (V), especially of at least one polyurethane polymer PUP, with at least one dialdimine A in the presence of a substoichiometric amount of water to give at least one compound VB of the formula (I), the aldimino groups undergoing hydrolysis in the dialdimine A react preferentially with the monomeric diisocyanates present in the polyisocyanate, especially a polyurethane polymer PUP. In this way, a large portion of the diisocyanate monomers originally present is converted, which greatly reduces the content of monomeric diisocyanates in the composition.

The composition with a low isocyanate monomer content thus obtained crosslinks on contact with moisture, for example from the air, to give a high molecular weight polymer. A great advantage in this crosslinking operation is the fact that the reaction products from the reaction of the polyisocyanate of the formula (V) with the dialdimine A are all at least difunctional in relation to the crosslinking reaction with water, which has the consequence of clean curing. If a polyisocyanate of the formula (V) were to be reacted in the manner described with a monofunctional compound, for example a monoalcohol without further reactive groups, a reduction in the monomer content would likewise be expected. However, the reaction products formed would all be monofunctional in relation to the crosslinking reaction with moisture, leading to the expectation of poorly crosslinked polymers with unsatisfactory properties on completion of curing. Another greatly advantageous fact is that the diisocyanate monomers converted in the process described are also incorporated into the crosslinked polymer in the course of curing of the composition.

The content of monomeric diisocyanates in the composition described is especially ≦1.0% by weight, preferably ≦0.5% by weight, based on the sum of the moisture-reactive constituents of the composition.

The composition described preferably contains at least 20% by weight of the compound VB of the formula (I) based on the sum of the compound VB of the formula (I) and of the polyisocyanate P.

The total weight of the compound of the formula (I) and of the polyisocyanate P is advantageously present in an amount of 5 to 100% by weight, preferably in an amount of 10 to 100% by weight, based on the overall composition.

The proportion of the polyisocyanate P in the composition described is preferably at least sufficiently great that the composition has at least as many isocyanate groups as aldimino groups, such that the action of moisture forms a high molecular weight polyurethane polymer.

The composition described may, in addition to at least one compound VB of the formula (I) having aldimino groups and at least one polyisocyanate P, optionally contain further assistants and additives. For this purpose, the following substances, for example, are suitable:

plasticizers, for example carboxylic esters such as phthalates, for example dioctyl phthalate, diisononyl phthalate or diisodecyl phthalate, adipates, for example dioctyl adipate, azelates and sebacates, organic phosphoric and sulfonic esters or polybutenes;

solvents;

inorganic and organic fillers, for example ground or precipitated calcium carbonates optionally coated with stearates, carbon blacks, especially industrially produced carbon blacks (referred to hereinafter as “carbon black”), barite (BaSO₄, also known as heavy spar), kaolins, aluminum oxides, aluminum hydroxides, silicas, especially high-dispersity silicas from pyrolysis processes, PVC powders or hollow spheres;

fibers, for example of polyethylene;

pigments, for example titanium dioxide or iron oxides;

catalysts which accelerate the hydrolysis of the aldimines, for example organic carboxylic acids such as benzoic acid, salicylic acid or 2-nitrobenzoic acid, organic carboxylic anhydrides such as phthalic anhydride, hexahydrophthalic anhydride and hexahydromethylphthalic anhydride, silyl esters of organic carboxylic acids, organic sulfonic acids such as methanesulfonic acid, p-toluenesulfonic acid or 4-dodecylbenzenesulfonic acid, sulfonic esters, other organic or inorganic acids, or mixtures of the aforementioned acids and acid esters;

catalysts which accelerate the reaction of the isocyanate groups, for example organotin compounds such as dibutyltin diacetate, dibutyltin dilaurate, dibutyltin dichloride, dibutyltin diacetylacetonate and dioctyltin dilaurate, bismuth compounds such as bismuth trioctoate and bismuth tris(neodecanoate), and compounds containing tertiary amino groups, such as 2,2′-dimorpholinodiethyl ether and 1,4-diazabicyclo[2.2.2]octane;

rheology modifiers, for example thickeners, for example urea compounds, polyamide waxes, bentonites or fumed silicas;

reactive diluents and crosslinkers, for example monomeric polyisocyanates such as MDI, TDI, mixtures of MDI and MDI homologs (polymeric MDI or PMDI), and oligomers of these polyisocyanates, especially in the form of iso-cyanurates, carbodiimides, uretonimines, biurets, allophanates or iminooxadiazinediones, adducts of monomeric polyisocyanates with short-chain polyols, and also adipic dihydrazide and other dihydrazides;

further latent hardeners with protected amino groups, for example ketimines, oxazolidines, enamines or other aldimines;

desiccants, for example molecular sieves, calcium oxide, high-reactivity isocyanates such as p-tosyl isocyanate, orthoformic esters, alkoxysilanes such as tetraethoxysilane, organoalkoxysilanes such as vinyltri-methoxysilane, and organoalkoxysilanes which have a functional group in the α position to the silane group;

adhesion promoters, especially organoalkoxysilanes, for example epoxysilanes, vinylsilanes, (meth)acryloylsilanes, isocyanatosilanes, carbamatosilanes, S-(alkylcarbonyl)mercaptosilanes and aldiminosilanes, and oligomeric forms of these silanes;

stabilizers against heat, light and UV radiation;

flame retardant substances;

surface active substances, for example wetting agents, leveling agents, devolatilizers or defoamers;

biocides, for example algicides, fungicides or substances which inhibit fungal growth.

It is advantageous to ensure that such additives do not impair the storage stability of the composition. This means that these additives must not trigger the reactions which lead to crosslinking, such as hydrolysis of the aldimino groups or crosslinking of the isocyanate groups, to a significant degree during storage. More particularly, this means that all of these additives should contain at most traces of water, if any. It may be advisable to chemically or physically dry certain additives before they are mixed into the composition.

The composition described preferably comprises, as well as at least one compound VB and at least one polyisocyanate P, at least one catalyst in the form of one of the organic acids mentioned, such as especially benzoic acid or salicylic acid, and/or in the form of one of the organometallic compounds mentioned, and/or in the form of one of the compounds containing tertiary amino groups mentioned.

The composition described is stored with exclusion of moisture. It is storage-stable, i.e. it can be stored with exclusion of moisture in a suitable package or arrangement, for example a vat, a hobbock, a bucket, a bag or a cartridge, over a period of several months up to one year and longer, without its application properties or its properties after curing changing to a degree relevant for the use thereof. Typically, the storage stability is determined via the measurement of the viscosity.

The aldimino groups of the compound VB and any further aldimino groups present have the property of being hydrolyzed on contact with moisture. The isocyanate groups present in the composition react in a formal sense with the amino groups released in the course of hydrolysis of the aldimino groups, which releases the corresponding aldehyde of the formula (VII a) or (VII b). Excess isocyanate groups in relation to the aldimino groups react with water present. As a result of these reactions, the composition cures to form a high molecular weight polymer; this process is also referred to as crosslinking. The reaction of the isocyanate groups with the aldimino groups being hydrolyzed need not necessarily proceed via the amino groups. It will be appreciated that reactions with intermediates of the hydrolysis of the aldimino groups to amino groups are also possible. For example, it is conceivable that aldimino groups being hydrolyzed will react directly with the isocyanate groups in the form of hemiaminals. In the case of a hotmelt adhesive composition, this brings about crosslinking, as a result of which the composition is no longer meltable. When exclusively aldimino groups proceeding from the particularly preferred odorless aldehydes of the formula (VII a) mentioned are present in the composition, the curing of the composition does not give rise to any troublesome odor, which is a great advantage or even an indispensable prerequisite for many applications, especially for applications in closed spaces, such as in the interior of buildings or of vehicles, or for large-area applications, for example floor coatings, or for applications at elevated temperature, for example hotmelt adhesives.

The water required for the curing reaction may originate from the air (air humidity), or else the composition can be contacted with a water-containing component, for example by spraying, or a water-containing component can be added to the composition in the course of application.

The composition cures on contact with moisture, without the formation of bubbles. The curing rate can be influenced via the type and amount of one or more optionally present catalysts and via the temperature which exists in the course of curing.

The composition described can be used for a wide variety of different purposes. For example, it is suitable as an adhesive for the adhesive bonding of various substrates, for example for adhesive bonding of components in the production of automobiles, rail vehicles, ships or other industrial goods, especially as a reactive hotmelt adhesive, as a sealant of all kinds, for example for sealing joints in construction, and as a coating or covering for various articles or variable substrates. Preferred coatings are protective paints, seals, protective coatings and primers. Among the coverings, particularly floor coverings should be mentioned as preferred. Such coverings are produced by pouring the composition typically onto the substrate and leveling it, where it cures to give a floor covering. For example, such floor coverings are used for offices, living areas, hospitals, schools, warehouses, garages and other domestic or industrial applications.

In a preferred embodiment, the composition described is used as a sealant or adhesive, especially as a reactive hotmelt adhesive.

The compositions described are particularly suitable for applications in which a low content of monomeric diisocyanates is required. These are especially applications in which the composition is sprayed, and applications in which the composition is applied at elevated temperature, for example as a hotmelt adhesive.

A further aspect of the present invention relates to a process for adhesive bonding a substrate S1 to a substrate S2, comprising the steps of:
i) applying one of the compositions described above to a substrate S1;
ii) contacting the applied composition with a substrate S2 within the open time of the composition;

or
i′) applying one of the compositions described above to a substrate S1 and to a substrate S2;
ii′) contacting the applied compositions with one another within the open time of the composition;

said substrate S2 consisting of the same material as or a different material than said substrate S1.

It is important here to ensure that the parts are joined within the open time, in order to ensure that the two joined parts are reliably adhesive bonded to one another.

In the application as a sealant, the composition is applied between the substrates S1 and S2, which is followed by the curing. Typically, the sealant is injected into a joint.

In both applications, the substrate S1 may be the same as or different than the substrate S2.

Suitable substrates S1 and/or S2 are, for example, inorganic substrates such as glass, glass ceramic, concrete, mortar, brick, tile, gypsum, and natural rocks such as granite or marble; metals or alloys such as aluminum, steel, nonferrous metals, galvanized metals; organic substrates such as wood, plastics such as PVC, polycarbonates, PMMA, polyesters, epoxy resins, polyurethanes (PU); coated substrates such as powder-coated metals or alloys; and paints and lacquers, especially automotive paints.

The substrates can be pretreated if required before the application of the adhesive or sealant. Such pretreatments include especially physical and/or chemical cleaning methods, for example grinding, sandblasting, brushing or the like, or treatment with detergents or solvents, or the application of an adhesion promoter, of an adhesion promoter solution or of a primer.

In the present document, a “primer” is understood to mean a composition suitable as an undercoat, which, as well as nonreactive volatile substances and optionally solid additives, comprises at least one polymer and/or at least one substance with reactive groups, and which is capable, when applied to a substrate, of curing to a solid, firmly adhering film in a layer thickness of typically at least 10 μm, the curing resulting either solely through the evaporation of the nonreactive volatile substances, for example solvents or water, or through a chemical reaction, or through a combination of these factors, and which builds up good adhesion to a layer applied subsequently, for example an adhesive or sealant.

The composition can be applied within a wide temperature range. For example, the composition can be applied at room temperature, as is typical of an elastic adhesive or a sealant. However, the composition can also be applied at lower and also at higher temperatures. The latter is advantageous especially when the composition comprises high-viscosity or meltable components, as are typically present in melt-applied adhesives, for example warm-melt adhesives or hotmelt adhesives. The application temperatures for warm-melts are, for example, in the range from 40 to 80° C., and in the case of hotmelts in the range from 85 to 200° C.

Thus, in the case of a warm-melt, the composition, prior to application, is heated to a temperature of 40° C. to 80° C., especially of 60° C. to 80° C., and is applied especially at this temperature in step i) or i′) of the above-described process. In the case of a hotmelt, the composition, prior to application, is heated to a temperature of 85° C. to 200° C., especially of 100° C. to 180° C., preferably of 120° C. to 160° C., and is applied especially at this temperature in step i) or i′) of the above-described process.

The above-described process for adhesive bonding, or for sealing, of the substrates S1 and S2 affords an adhesive-bonded or sealed article. Such an article may be an article from the building sector, transport sector, furniture sector, textile sector or packaging sector. For instance, such an article may be a built structure, especially a built structure in construction or civil engineering, or a mode of transport, for example a water or land vehicle, especially an automobile, a bus, a truck, a train or a ship, or an installable component thereof.

Especially if the composition is used as an adhesive for elastic adhesive bonds, it preferably has a pasty consistency with structurally viscous properties. Such an adhesive is applied to the substrate by means of a suitable apparatus, preferably in the form of a bead, which may have an essentially round or triangular cross-sectional area. Suitable methods for applying the adhesive are, for example, application from commercial cartridges, which are operated manually or by means of compressed air, or from a vat or hobbock by means of a delivery pump or of an extruder, if appropriate by means of an application robot. An adhesive with good application properties has a long shelf life and short stringing. In other words, it remains in the form applied after application, i.e. does not flow apart, and forms only a very short thread, if any, after the application unit has been moved away, such that the substrate is not soiled.

The composition crosslinks rapidly with a small amount of water and without the formation of bubbles. For an application, for example, as an adhesive, this means that an adhesive bond is already stressable to a certain degree at an early stage, even if only a relatively small amount of water from the environment has come into contact with the composition as yet. This is a great advantage in industrial manufacture, for example in the assembly of vehicles, since components attached by adhesive bonding should be kept in position by the adhesive bond even after a relatively short time, and the adhesive-bonded object, as a result, can be moved and processed further without further fixing.

The cured composition which is obtained by the reaction of an above-described composition with water, especially in the form of air humidity, is notable for excellent properties. It possesses, for example, a high extensibility and a high tensile strength. Its modulus of elasticity varies as a function of the components used to produce the composition, for example the polyols, polyisocyanates or diamines, and can thus be adjusted to the requirements of a particular application, for example to high values for adhesives or to low values for sealants.

In a particularly preferred embodiment, the composition described is a reactive hotmelt adhesive composition. In this case, both the polyisocyanate which has (u+v) isocyanate groups and represents Q of the compound VB and the polyisocyanate P are a room temperature solid polyurethane polymer PUP1 having (u+v) isocyanate groups, as has already been described in detail above.

Such hotmelt adhesive compositions are advantageously unfilled. It is advantageous that, in such hotmelt adhesive compositions, the total weight of the compound of the of the formula (I) and of the polyisocyanate P, especially of the room temperature solid polyurethane polymer PUP1, is present 40 to 100% by weight, especially 75 to 100% by weight, preferably 80 to 100% by weight, based on the overall composition.

The hotmelt adhesive composition described has a surprisingly low content of monomeric diisocyanates. This is particularly advantageous for a hotmelt adhesive, since monomeric diisocyanates outgas in the course of application and, being irritant, sensitizing or toxic substances, can constitute a health risk to the user. The content of monomeric diisocyanates in the hotmelt adhesive composition described is especially ≦1.0% by weight, preferably ≦0.5% by weight, based on the sum of the moisture-reactive constituents of the composition.

Particularly suitable optional further constituents of such a hotmelt adhesive composition are nonreactive thermoplastic polymers, for example homo- or copolymers of unsaturated monomers, especially from the group comprising ethylene, propylene, butylene, isobutylene, isoprene, vinyl acetate or higher esters thereof, and (meth)acrylate, particularly suitable optional further constituents being ethylene-vinyl acetate copolymers (EVA), atactic poly-α-olefins (APAO), polypropylenes (PP) and polyethylenes (PE).

For the mode of action of a reactive hotmelt adhesive, it is important that the adhesive is meltable, which means that it has a sufficiently low viscosity at the application temperature to be applicable, and that it very rapidly builds up a sufficient adhesive strength in the course of cooling, even before the crosslinking reaction with air humidity is complete (initial strength). It has been found that the hotmelt adhesive composition described, at the customary application temperatures in the range from 85° C. to 200° C., typically 120° C. to 160° C., has a readily manageable viscosity, and that a good adhesive strength builds up sufficiently rapidly in the course of cooling.

On application, the hotmelt adhesive composition described comes into contact with moisture, especially in the form of air humidity. In parallel to the physical hardening owing to solidification in the course of cooling, chemical crosslinking with moisture also sets in, principally by virtue of the aldimino groups present being hydrolyzed by moisture and reacting rapidly with isocyanate groups present in the manner already described. Excess isocyanate groups likewise crosslink with moisture in a known manner.

The moisture required for the chemical reaction may originate from the air (air humidity), or else the composition can be contacted with a water-containing component, for example by painting or by spraying, or a water-containing component can be added to the composition in the course of application, for example in the form of an aqueous paste which is mixed in, for example by means of a static mixer.

In the course of crosslinking with moisture, the hotmelt adhesive composition described exhibits a greatly reduced tendency to form bubbles, since little carbon dioxide or none whatsoever—according to the stoichiometry—forms in the course of crosslinking as a result of the presence of aldimino groups.

Suitable substrates S1 and/or S2 in the process for adhesive bonding by means of the hotmelt adhesive composition described are especially plastics, organic materials such as leather, fabrics, paper, wood, resin-bound wood materials, resin-textile composite materials, glass, porcelain, ceramic and metals and metal alloys, especially coated or power-coated metals and metal alloys.

Suitable plastics in this context are especially polyvinyl chloride (PVC), acrylonitrile-butadiene-styrene copolymers (ABS), SMC (sheet molding composites), polycarbonate (PC), polyamide (PA), polyester, polyoxymethylene (POM), polyolefins (PO), especially polyethylene (PE), polypropylene (PP), ethylene/propylene copolymers (E PM) and ethylene/propylene-diene terpolymers (EPDM), preferably surface-plasma-, -corona- or -flame-treated PP or PE.

The thickness of the adhesive layer (adhesive bond thickness) in the case of hotmelt adhesives is typically 10 micrometers or more. The adhesive bond thickness is especially between 10 micrometers and 20 millimeters, in particular between 80 micrometers and 500 micrometers. In the case of thick layers, the crosslinking, caused by the slow water diffusion, however, is typically very slow.

The hotmelt adhesive composition described is especially used in an industrial manufacturing process. This results especially in articles from the transport sector, furniture sector or textile sector. A preferred transport sector is the automotive sector.

Examples of such articles are water or land vehicles, such as automobiles, buses, trucks, trains or ships; automotive interior trim parts, such as roof linings, sun visors, instrument panels, door side parts, parcel shelves and the like; wood fiber materials from the showers and baths sector; decorative films for furniture, membrane films with textiles such as cotton, polyester films in the clothing sector or textiles with foams for automotive trim.

On the other hand, such articles are especially articles from the packaging sector.

The hotmelt adhesive composition described has a series of advantages over the prior art.

They have a greatly reduced content of monomeric diisocyanates and lead as a result, when they are used, to a greatly reduced exposure of the user to harmful diisocyanate vapors. With the compositions described, hotmelt adhesive compositions based on commercial, readily obtainable diisocyanates such as 4,4′-MDI or IPDI and having an exceptionally low content of monomeric diisocyanates are obtainable.

Moreover, the hotmelt adhesive composition described possesses a high crosslinking rate, even though it contains only slow-reacting aliphatic isocyanate groups, for example those of IPDI or H₁₂MDI. Prior art reactive hotmelt adhesives based on purely aliphatic diisocyanates generally have such a low crosslinking rate that they are unusable for many applications.

Moreover, the hotmelt adhesive composition described exhibits a greatly reduced tendency to form bubbles, since no carbon dioxide is formed in the crosslinking reaction of isocyanate groups with aldimino groups being hydrolyzed, in contrast to the crosslinking of isocyanate groups with moisture.

In addition to these advantages, the hotmelt adhesive compositions described exhibit similarly good properties to the prior art systems, specifically rapid adhesive strength, good thermal stability and high final strength coupled with good extensibility, the final mechanical properties being adjustable to the requirements of an adhesive application within a very wide range.

A particularly significant aspect of the present invention relates to a process for reducing the content of monomeric diisocyanates in polyurethane polymers having isocyanate groups or in oligomeric polyisocyanates or in compositions which comprise polyurethane polymers having isocyanate groups or oligomeric polyisocyanates, by reacting them with at least one polyaldimine A′ of the formula (IV a′) or (IV b′) in the presence of a substoichiometric amount of water, especially less than 0.5 mol of water per equivalent of aldimino groups. This process is thus not limited to the above-described “asymmetric” polyaldimines A of the formula (IV a) or (IV b), and it has instead been found that it leads to the desired reduction in the content of monomeric diisocyanates even in the case of nonasymmetric polyaldimines.

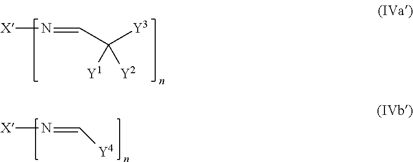

In the formulae (IV a′) and (IV b′), X′ is the radical of a polyamine DA′ with n primary amino groups after the removal of these n amino groups, and n is from 2 to 6, especially 2 or 3, preferably 2. Y¹, Y², Y³ and Y⁴ are each as already defined for formula (I).

The polyamines DA′ have n primary amino groups. Apart from these n amino groups, the polyamines DA′ are free of moieties which are reactive with isocyanate groups; more particularly, they have no hydroxyl groups, no secondary amino groups and no mercapto groups.

Suitable polyamines DA′ are, in addition to the “asymmetric” diamines DA already mentioned, for example, the following diamines and triamines:

aliphatic diamines such as ethylenediamine, 1,3-propanediamine, 2-methyl-1,2-propanediamine, 2,2-dimethyl-1,3-propanediamine, 1,4-butanediamine, 1,5-pentanediamine, 1,6-hexamethylenediamine, 1,7-heptanediamine, 1,8-octanediamine, 1,9-nonanediamine, 1,10-decanediamine, 1,11-undecanediamine, 1,12-dodecanediamine, methylbis(3-aminopropyl)-amine, 1,5-diamino-2-methylpentane (MPMD), 2,5-dimethyl-1,6-hexamethylenediamine;

cycloaliphatic diamines such as 1,3- and 1,4-diaminocyclohexane, bis-(4-aminocyclohexyl)methane, bis(4-amino-3-methylcyclohexyl)methane, bis-(4-amino-3-ethylcyclohexyl)methane, bis(4-amino-3,5-dimethylcyclohexyl)-methane, bis(4-amino-3-ethyl-5-methylcyclohexyl)methane (M-MECA), 2-methyl-1,3-diaminocyclohexane, 1,3- and 1,4-bis(aminomethyl)cyclo-hexane, 2,5(2,6)-bis(aminomethyl)bicyclo[2.2.1]heptane (NBDA), 3(4),8(9)-bis(aminomethyl)tricyclo[5.2.1.0^{2,6}]decane, 3,9-bis(3-aminopropyl)-2,4,8,10-tetraoxaspiro[5.5]undecane, 1,3- and 1,4-xylylenediamine;

aliphatic diamines containing ether groups, such as bis(2-aminoethyl) ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine and higher oligomers of these diamines;

polyoxyalkylenediamines, which are typically products from the amination of polyoxyalkylenediols and are, for example, obtainable under the Jeffamine® trade name (from Huntsman Chemicals), under the Polyetheramine name (from BASF) or under the PC Amine® name (from Nitroil), for example Jeffamine® D-230, Jeffamine® D-400, Jeffamine® D-2000, Jeffamine® D-4000, Jeffamine® XTJ-511, Jeffamine® ED-600, Jeffamine® ED-900, Jeffamine® ED-2003, Jeffamine® XTJ-568, Jeffamine® XTJ-569, Jeffamine® XTJ-523, Jeffamine® XTJ-536, Jeffamine® XTJ-542, Jeffamine® XTJ-559; Polyetheramine D 230, Polyetheramine D 400 and Polyetheramine D 2000, PC Amine® DA 250, PC Amine® DA 400, PC Amine® DA 650 and PC Amine® DA 2000;

aliphatic triamines such as 4-aminomethyl-1,8-octanediamine, 1,3,5-tris-(aminomethyl)benzene, 1,3,5-tris(aminomethyl)cyclohexane;

polyoxyalkylenetriamines, which are typically products from the amination of polyoxyalkylenetriols and are, for example, obtainable under the Jeffamine® trade name (from Huntsman Chemicals), under the Polyetheramine trade name (from BASF) or under the PC Amine® name (from Nitroil), for example Jeffamine® T-403, Jeffamine® T-5000; Polyetheramine T403, Polyetheramine T5000; and PC Amine® TA 403, PC Amine® TA 5000.

The polyamine DA′ is particularly advantageously a diamine, preferably an asymmetric diamine DA as described above.

The surprising reduction in the content of monomeric diisocyanates is probably achieved by virtue of the fact that, in this reaction of at least one polyurethane polymer having isocyanate groups or of an oligomeric polyisocyanate with at least one polyaldimine A′ in the presence of a substoichiometric amount of water, the aldimino groups of the polyaldimine A′ undergoing hydrolysis react preferentially with the polyurethane polymer having isocyanate groups or the monomeric diisocyanates present in the oligomeric polyisocyanate. In this way, a large portion of the diisocyanate monomers originally present is converted.

### EXAMPLES

Description of the Test Methods

Infrared spectra were measured on an FT-IR 1600 instrument from Perkin-Elmer (horizontal ATR analysis unit with ZnSe crystal). Liquid samples were applied undiluted as a film; solid samples were dissolved in CH₂Cl₂. The absorption bands are reported in wavenumbers (cm⁻¹) (measurement window: 4000-650 cm⁻¹).

¹H NMR spectra were measured on a Bruker DPX-300 spectrometer at 300.13 MHz. The chemical shifts δ are reported in ppm relative to tetramethylsilane (TMS), coupling constants J are reported in Hz. True coupling patterns and pseudo coupling patterns were not distinguished.

The viscosity was measured at the temperature specified on a thermostated Physica UM cone-plate viscometer (cone diameter 20 mm, cone angle 1°, cone tip-plate distance 0.1 mm, shear rate 10 to 1000 s⁻¹).

The amine content of the dialdimines prepared, i.e. the content of protected amino groups in the form of aldimino groups, was determined titrimetrically (with 0.1N HClO₄ in glacial acetic acid, using crystal violet), and is always reported in mmol N/g.

The content of monomeric diisocyanates was determined by means of HPLC (detection by means of a photodiode array; 0.04 M sodium acetate/acetonitrile as mobile phases) and is reported in % by weight based on the overall composition.

The tensile strength was determined based on DIN 53504, on dumbbells with a thickness of 1 mm and a length of 75 mm (central element length 30 mm, central element width 4 mm). To produce the dumbbells, an adhesive film of thickness 1 mm was prepared (application temperature of the adhesive 130° C.), from which the dumbbells were punched out, which were then stored at 23° C. and 50% relative air humidity over the time specified.

a) Preparation of Dialdimines

Dialdimine A-1

A round-bottom flask was initially charged under a nitrogen atmosphere with 55.0 g (0.19 mol) of distilled 2,2-dimethyl-3-lauroyloxypropanal. With vigorous stirring, 15.6 g (0.18 mol of N) of 1-amino-3-aminomethyl-3,5,5-trimethylcyclo-hexane (=isophoronediamine, IPDA; Vestamin® IPD, Degussa; amine content 11.68 mmol N/g) were added slowly from a dropping funnel, in the course of which the mixture heated up and became increasingly cloudy. Thereafter, the volatile constituents were removed under reduced pressure (10 mbar, 80° C.). Yield: 67.1 g of a clear colorless oil with an amine content of 2.73 mmol N/g and a viscosity of 190 mPa·s at 20° C.

IR: 2952, 2922, 2852, 2819sh, 1738 (C═O), 1666 (C═N), 1464, 1418, 1394, 1378, 1364, 1350, 1298, 1248, 1236sh, 1158, 1112, 1048, 1020, 1000, 938, 928, 910, 894, 868, 772, 722.

¹H NMR (CDCl₃, 300 K): δ 7.59 and 7.57 (2×s, total 1 H, CH═N ([isomers]), 7.47 (s, 1 H, CH═N), 4.03 and 4.01 (2×s, 2×2 H, C(CH₃)₂—CH₂—O), 3.37 (m, 1 H, N—CH^{Cy}), 3.08 (dd, 2 H, J≈11.1, N—CH₂—C^{Cy}), 2.30 (t, 4 H, J≈7.5, OC(O)—CH₂—CH₂), 1.61 (m, 4 H, OC(O)—CH₂—CH₂), 1.60-0.85 (m, 65 H, remaining CH).

Dialdimine A-2

A round-bottom flask was initially charged under a nitrogen atmosphere with 55.0 g (0.19 mol) of distilled 2,2-dimethyl-3-lauroyloxypropanal. With vigorous stirring, 9.4 g (0.18 mol of N) of 1,3-diaminopentane (DAMP; Dytek® EP Diamine, Invista; amine content 19.42 mmol N/g) were added slowly from a dropping funnel, in the course of which the mixture heated up and became increasingly cloudy. Thereafter, the volatile constituents were removed under reduced pressure (10 mbar, 80° C.). Yield: 60.9 g of a clear, pale yellow oil with an amine content of 3.01 mmol N/g and a viscosity of 50 mPa·s at 20° C. IR: 2955sh, 2922, 2868sh, 2852, 1737 (C═O), 1666 (C═N), 1466, 1419, 1394, 1373, 1346, 1300, 1248, 1233, 1159, 1112, 1057, 1019, 1000, 935, 884, 769br, 722.

Dialdimine A-3

A round-bottom flask was initially charged under a nitrogen atmosphere with 15.0 g (0.175 mol of N) of 1-amino-3-aminomethyl-3,5,5-trimethylcyclohexane (=isophoronediamine, IPDA; Vestamin® IPD, Degussa; amine content 11.68 mmol N/g). With vigorous stirring, 26.0 g (0.180 mol) of distilled 3-acetoxy-2,2-dimethylpropanal were added slowly from a dropping funnel, in the course of which the mixture heated up significantly. Thereafter, the volatile constituents were removed under reduced pressure (10 mbar, 80° C.). Yield: 37.3 g of a clear colorless oil with an amine content of 4.69 mmol N/g and a viscosity of 820 mPa·s at 20° C.

IR: 2954, 2931, 2925, 2906, 2869, 2846, 2821, 1740 (C═O), 1666 (C═N), 1470, 1462, 1440sh, 1394, 1374, 1365sh, 1232, 1038, 1008, 986, 971, 940sh, 926, 912, 895, 866, 844, 798.

b) Preparation of Polyurethane Polymers

Polyurethane Polymer 1

515 g of Dynacoll® 7360 polyol (Degussa; crystalline polyesterdiol, OH number 32 mg KOH/g, acid number approx. 2 mg KOH/g) and 84 g of 4,4′-methylenediphenyl diisocyanate (MDI; Desmodur® 44 MC L, Bayer) was converted by known methods at 80° C. to an NCO-terminated polyurethane polymer. The room temperature solid reaction product had a content of free isocyanate groups determined by titrimetric means of 1.95% by weight.

Polyurethane Polymer 2

232 g of Dynacoll® 7360 polyol (Degussa; crystalline polyesterdiol, OH number 32 mg KOH/g, acid number approx. 2 mg KOH/g), 155 g of Dynacoll® 7110 polyol (Degussa; amorphous polyesterdiol, OH number 52 mg KOH/g, acid number approx. 10 mg KOH/g) and 62 g of isophorone diisocyanate (IPDI; Vestanat® IPDI, Degussa) were converted by known methods at 130° C. to an NCO-terminated polyurethane polymer. The room temperature solid reaction product had a content of free isocyanate groups determined by titrimetric means of 2.16% by weight.

c) Production of Hotmelt Adhesives

Examples 1 to 3 and comparative example 4

For each example, the particular constituents according to table 1 were heated to 100° C. and weighed under a nitrogen atmosphere in the parts by weight specified into a screwtop polypropylene cup, and mixed by means of a centrifugal mixer (SpeedMixer™ DAC 150, FlackTek Inc.; 1 min at 3000 rpm). Immediately thereafter, 0.19 g of water mixed with 0.2 g of Plurafac® LF 132 (BASF; terminally etherified fatty alcohol alkoxylate) was added to the mobile mixture thus obtained and mixed in by means of a centrifugal mixer (1 min at 3000 rpm). Immediately thereafter, the mixture was transferred into an internally coated aluminum tube which was sealed airtight.

TABLE 1
Composition of examples 1 to 3 and of comparative example 4.

| | Example | | | |
|---|---|---|---|---|
| | | | | 4 |
| | 1 | 2 | 3 | (comparative) |
| Polyurethane polymer 1 | 50.0 | 50.0 | 50.0 | 50.0 |
| Dialdimine | A-1, | A-2, | A-3, | — |
| | 7.67 | 6.93 | 4.46 | |

In examples 1 to 3, the ratio between the isocyanate groups in the polyurethane polymer 1 and the aldimino groups of the dialdimines is 1.0/0.9; the ratio between water and the aldimino groups of the dialdimines is 0.5/1.0.

The hotmelt adhesives of examples 1 to 3 and of comparative example 4 produced in this way were homogeneous and mobile, and were tested for viscosity, content of monomeric 4,4′-methylenediphenyl diisocyanate (4,4′-MDI) and tensile strength. The results are shown in table 2.

TABLE 2
Properties of examples 1 to 3 and of comparative example 4.

| | Example | | | |
|---|---|---|---|---|
| | | | | 4 |
| | 1 | 2 | 3 | (comparative) |
| Content of monomeric | 0.23 | 0.19 | 0.33 | 1.57 |
| 4,4′-MDI [% by weight] | | | | |
| Viscosity at 130° C. [Pa · s] | 38 | 130 | 35 | 7.1 |
| Tensile strength | 8.8 | 6.7 | 5.8 | 18.7 |

### Example 5 and Comparative Example 6

For each example, the particular constituents according to table 3 were heated to 130° C. and weighed under a nitrogen atmosphere in the parts by weight specified into a screwtop polypropylene cup and mixed by means of a centrifugal mixer (SpeedMixer™ DAC 150, FlackTek Inc.; 1 min at 3000 rpm). Immediately thereafter, 0.21 g of water mixed with 0.2 g of Plurafac® LF 132 (BASF; terminally etherified fatty alcohol alkoxylate) was added to the mobile mixture thus obtained, and mixed in by means of a centrifugal mixer (1 min at 3000 ppm). Immediately thereafter, the mixture was transferred into an internally coated aluminum tube which was sealed airtight.

TABLE 3
Composition of example 5 and of comparative example 6.

Example

| | | 6 |
|---|---|---|
| | 5 | (comparative) |
| Polyurethane polymer 2 | 50.0 | 50.0 |
| Tin catalyst^{a} | 0.01 | 0.01 |
| Dialdimine | A-1, | — |
| | 8.49 | |

^{a}Dibutyltin dilaurate.

In example 5, the ratio between the isocyanate groups in the polyurethane polymer 2 and the aldimino groups of the dialdimine A-1 is 1.0/0.9; the ratio between water and the aldimino groups of the dialdimine A-1 is 0.5/1.0.

The hotmelt adhesives thus produced were then tested for viscosity before and after storage, content of monomeric isophorone diisocyanate (IPDI; sum of cis and transisomers) and tensile strength. The results are shown in table 4.

TABLE 4
Properties of example 5 and of comparative example 6.

Example

| | | 6 |
|---|---|---|
| | 5 | (comparative) |
| Content of monomeric IPDI | 0.37 | 1.36 |
| [% by weight] | | |
| Viscosity at 130° C. [Pa · s] | 2.4 | 3.1 |
| before storage | | |
| Viscosity at 130° C. [Pa · s] | 4.0 | 4.3 |
| after storage^{a} | | |
| Tensile strength | 6.1 | 24.3 |

^{a}Over 12 days at 70° C.

What is claimed is:

1. A process for preparing a compound of the formula (I) having isocyanate groups and aldimino groups, comprising:
reacting at least one dialdimine A of the formula (IV a) or (IV b) with at least one polyisocyanate of the formula (V) in the presence of a substoichiometric amount of water, 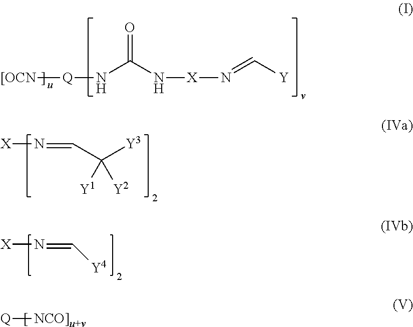
wherein:
Q is a radical of a polyisocyanate after removal of u +v isocyanate groups;
u is 1 or 2;
v is 1 or 2;
Y is a radical of the formula (I a) or (I b) 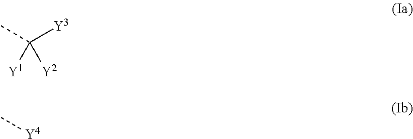
Y¹ and Y² are each independently a monovalent hydrocarbon radical having 1 to 12 carbon atoms, or together are a divalent hydrocarbon radical having 4 to 20 carbon atoms and is part of an optionally substituted carbocyclic ring having 5 to 8 carbon atoms;
Y³ is a monovalent hydrocarbon radical which optionally has at least one heteroatom; and
Y⁴ is a substituted or unsubstituted aryl or heteroaryl group which has a ring size of 5 to 8 atoms, or is 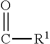
where R¹ is a hydrogen atom or an alkoxy group, or is a substituted or unsubstituted alkenyl or arylalkenyl group having at least 6 carbon atoms; and
X is a radical of a diamine DA after removal of two primary amino groups of the diamine DA, at least one of the two primary amino groups of the diamine DA being an aliphatic amino group and the two primary amino groups of the diamine DA being different from one another either in the number of hydrogen atoms on the carbon atoms (C_{α}) in the α position to the particular amino group by at least one, or in the number of hydrogen atoms on the carbon atoms (C_{β}) in the β position to the particular amino group by at least two.

2. The process as claimed in claim 1, wherein a ratio between water and the aldimino groups of the dialdimine A is at most 0.5 mol of water per equivalent of aldimino groups.

3. The process as claimed in claim 2, wherein the ratio between water and the aldimino groups of the dialdimine A is between 0.3 and 0.5 mol of water per equivalent of aldimino groups.

4. The process as claimed in claim 1, wherein a ratio of the number of isocyanate groups of the polyisocyanate of the formula (V) to the number of aldimino groups of the dialdimine A of the formula (IV a) or (IV b) is at least 1.

5. The process as claimed in claim 4, wherein the ratio of the number of isocyanate groups of the polyisocyanate of the formula (V) to the number of aldimino groups of the dialdimine A of the formula (IV a) or (IV b) is from 1 to 5.

6. A process for reducing the content of monomeric diisocyanates in polyurethane polymers having isocyanate groups or in oligomeric polyisocyanates or in compositions which comprise polyurethane polymers having isocyanate groups or oligomeric polyisocyanates, comprising:
reacting at least one of the polyurethane polymers having isocyanate groups or oligomeric polyisocyanates with at least one polyaldimine A′ of the formula (IV a′) or (IV b′) in the presence of a substoichiometric amount of water, 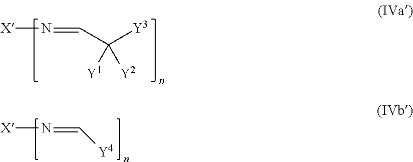
wherein:
Y¹ and Y² are each independently a monovalent hydrocarbon radical having 1 to 12 carbon atoms, or together are a divalent hydrocarbon radical having 4 to 20 carbon atoms and being part of an optionally substituted carbocyclic ring having 5 to 8 carbon atoms;
Y³ is a monovalent hydrocarbon radical, optionally having at least one heteroatom; and
Y⁴ is a substituted or unsubstituted aryl or heteroaryl group having a ring size of 5 to 8 carbon atoms, or is 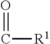
where R¹ is a hydrogen atom or an alkoxy group, or is a substituted or unsubstituted alkenyl or arylalkenyl group having at least 6 carbon atoms;
X is a radical of a polyamine DA′ after removal of n primary amino groups; and
n is from 2 to 6.

7. The process as claimed in claim 6, wherein Y³ is a radical of the formula (II) or (III) 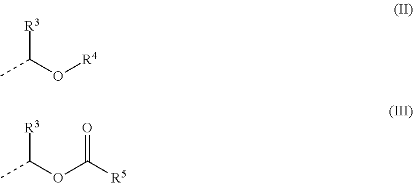
wherein:
R³ is a hydrogen atom or an alkyl or arylalkyl group;
R⁴ is a hydrocarbon radical which has 1 to 30 carbon atoms and optionally contains heteroatoms; and
R⁵ is a hydrogen atom, or is a linear or branched alkyl radical having 1 to 30 carbon atoms, optionally with cyclic components and optionally with at least one heteroatom, or is a mono- or polyunsaturated, linear or branched hydrocarbon radical having 5 to 30 carbon atoms, or is an optionally substituted aromatic or heteroaromatic 5- or 6-membered ring.

8. The process as claimed in claim 6, wherein the polyamine DA′ is a diamine with two primary amino groups and these two amino groups differ from one another either in the number of hydrogen atoms on the carbon atoms (C_{α}) in the α position to the particular amino group by at least one, or in the number of hydrogen atoms on the carbon atoms (C_{β}) in the β position to the particular amino group by at least two.

9. The process as claimed in claim 6, wherein the polyamine DA′ is selected from the group consisting of 1,2-propanediamine, 2-methyl-1,2-propanediamine, 1,3-butanediamine, 1,3-diaminopentane (DAMP), 4-aminoethylaniline, 4-aminomethylaniline, 4-[(4-aminocyclohexyl)methyl]aniline, 2-aminoethylaniline, 2-aminomethylaniline, 2-[(4-aminocyclohexyl)methyl]aniline, 4-[(2-aminocyclohexyl)methyl]aniline, 2,2,4-trimethylhexamethylenediamine (TMD), 1,5-diamino-2-butyl-2-ethylpentane, 1-amino-3-aminomethyl-3,5,5-trimethylcyclohexane (=isophoronediamine=PDA) and 1,4-diamino-2,2,6-trimethylcyclohexane (TMCDA).

10. The process as claimed in claim 6, wherein the reaction takes place in the presence of less than 0.5 mol of water per equivalent of aldimino groups.

11. The process as claimed in claim 6, wherein n is 2.

12. The process as claimed in claim 6, wherein Y³ is a radical of the formula (III).

13. The process as claimed in claim 6, wherein R³ is a hydrogen atom.
